(19) United States
(12) Patent Application Publication
(10) Pub. No.: US 2020/0155002 A1
(43) Pub. Date: May 21, 2020

(54) WIRELESS CARDIAC MONITORING SYSTEM

(71) Applicant: SABIC GLOBAL TECHNOLOGIES B.V., Bergen op Zoom (NL)

(72) Inventors: Manish NANDI, Malvern, PA (US); Ashir P. THAKORE, Novi, MI (US)

(21) Appl. No.: 16/604,381

(22) PCT Filed: Apr. 11, 2018
(86) PCT No.: PCT/US2018/027062

§ 371 Date: Oct. 10, 2019

Related U.S. Application Data
(60) Provisional application No. 62/484,154, filed on Apr. 11, 2017.

(51) Int. Cl. A61B 5/00; A61B 5/0408

(52) U.S. Cl. CPC A61B 5/0024; A61B 5/0408; A61B 2562/0215; A61B 5/0006; A61B 2560/045; A61B 5/6823

(57) ABSTRACT

A cardiac monitoring system includes: a plurality of wireless electrode patches, each of the patches including a sensor to detect a characteristic of a subject, a wireless transmitter to transmit a signal of the detected characteristic, and a power module to supply electricity to one or more of the sensor and transmitter; and a wireless receiver to communicate with each of the wireless electrode patches to receive the signal indicative of the detected characteristic.

### TECHNICAL FIELD

The disclosure concerns a cardiac monitoring system and more particularly, a wearable, ambulatory electrocardiograph system utilizing multiple electrode patches and a receiver.

### BACKGROUND

An electrocardiograph (ECG) system monitors and measures heart electrical activity in a subject over a period of time. Such measurement occurs via electrodes placed on the surface of the skin of the particular subject.

Traditionally, ECG systems utilize a 12 lead system, with 10 electrode leads placed at various anatomical positions on a subject to provide a complete structural and functional three-dimensional analysis of the heart. The electrode leads are used to produce electrical signals corresponding to the electrical activity generated by the heart of the subject. Such signals are generally transmitted via wiring or cable to a display which processes the signal information and converts such data into a comprehensible format for review by a health care professional.

Health care professionals have used ECG systems to monitor a subject's heart activity for years. Presently, there are a number of distinct systems that use ECG signals to monitor a subject's heart activity. These systems are not generally user-friendly, comfortable or portable, and are often cumbersome and visible to other individuals. Avoiding visibility of the system is important due to the stigma and potential embarrassment of the subject wearing the monitoring system. Thus, there exists a need to provide a cardiac monitoring system that is comfortable and portable, that may be worn under clothes without a pronounced or unnatural appearance, that produces quality and reliable data relating to the heart activity of a subject, and that uses fewer electrode patches and no wires relative to a traditional 12-lead wired ECG.

### SUMMARY

The present disclosure, in an aspect, provides a medical monitoring system comprising a plurality of wireless electrode patches, each of the wireless electrode patches comprising a sensor configured to detect a medical characteristic of a subject, a wireless module configured to transmit a signal indicative of the detected medical characteristic, and a power module configured to supply electrical energy to one or more of the sensor and the wireless module; and a wireless receiver configured to communicate with each of the wireless electrode patches to receive at least the signal indicative of the detected medical characteristic.

### DETAILED DESCRIPTION

The present disclosure can be understood more readily by reference to the following detailed description of the disclosure and the Examples included therein.

Before the present articles, systems, devices, and/or methods are disclosed and described, it is to be understood that they are not limited to specific synthetic methods unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

All publications mentioned herein are incorporated herein by reference to, for example, disclose and describe the methods and/or materials in connection with which the publications are cited.

### Definitions

It is also to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural referents unless the context clearly dictates otherwise. Thus, for example, reference to “an electrode patch” includes the singular patch or two or more electrode patches.

As used herein, the term “combination” is inclusive of different components working together, though not necessarily joined physically. Thus, for example, reference to a “combination of parts” includes, but is not limited to, the cooperation of an electrode patch, a wireless transmitter communication module, and a power module.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

I. Wireless Cardiac Monitoring System

In an aspect, the present disclosure pertains to a wireless cardiac monitoring system (e.g., an ambulatory ECG system) comprising a plurality of wireless electrode patches, each of the wireless electrode patches comprising a sensor configured to detect a medical characteristic of a subject, a wireless module configured to transmit a signal indicative of the detected medical characteristic, and a power module configured to supply electrical energy to one or more of the sensor and the wireless module; and a wireless receiver configured to communicate with each of the wireless electrode patches to receive at least the signal indicative of the detected medical characteristic.

In various aspects, the system of the present disclosure further comprises greater than three to five small wireless electrode patches. In other aspects, the system of the present disclosure may include locating the plurality of lead patches at different locations on the torso and limbs of the subject.

A. Wireless Electrode Patches

In one aspect, the wireless cardiac monitoring system comprises at least three wireless electrode patches. Each of the at least three wireless patches may be selectively placed at different locations on the torso or limbs of a subject. An electrode patch may comprise a thermoplastic substrate. In an aspect, a flexible silicone thermoplastic substrate may be used to encapsulate the electrode patch. In another aspect, a silver-silver chloride electrode may be used as a reference electrode. In this aspect, the silver-silver electrode may be used in electrochemical measurements. The silver-silver chloride electrode may comprise a silver wire that is coated by a layer of silver chloride so as to form an encapsulation of the silver wire. At one end of the electrode, a permeable body permits exposure between the surface and area to be measured and the silver chloride electrolyte. To transmit the measured data, an insulated lead wire may connect the silver wire to one or more measuring instruments. The lead patches may vary in size due to the size of a battery to be used. Such a lead patch may vary in size from about 0.5 inches (in) diameter to about 2 in diameter. Given the relatively diminutive size of the wireless electrode patches, the overall form factor and comfort of the subject will be greatly enhanced. In certain aspects, heuristics, learning models such as machine learning, or trial and error may be used to define a placement of a minimal number of electrodes on the patient's body, yet still be able to collect a full ECG reading of the patient.

In an aspect, an electrode patch comprises an electrode sensor, a wireless transmitter module for communication, and a power module.

The electrode patch functions as a conductor in collecting electrical signals from the body of the subject and transmitting those signals to a wireless receiver. More specifically, the electrode patch collects and transmits electrical signals from the heart of the subject.

In an aspect, each electrode patch may be labeled by name so as to avoid improper placement of the electrode patches on the body of the subject. In a further aspect, each electrode patch may be coded by color so as to avoid improper placement of the electrode patches on the body of the subject. In an aspect, each electrode patch may be labeled and coded by color so as to avoid improper placement of the electrode patches on the body of the subject.

Notably, the electrode patches must be located on the body of a subject with sufficient space between each patch so as to prevent electrical arcing across the electrode patches and potential injury to the subject and/or health care professional.

B. Electrode Sensor

Each individual wireless electrode patch contains an electrode sensor designed to detect electrical signals from each contraction, or beat of the heart of a subject.

Electrical activity of the heart begins with spontaneous generation of an action potential by the Sinoatrial (SA) node. Such action potential transmits through the right atrium of the heart, then through Bachmann's bundle and the left atrium of the heart. This transmission activates the myocardium, or muscle cells of the atria and causes contraction of the upper chambers of the heart, and is seen as a P wave on an electrocardiograph (ECG). Such electrical activity spread through the atria travel through intermodal tracts from the SA node to the Atrioventricular (AV) node. A delay, or PR interval on an ECG, between contraction of the atria and the ventricles of the heart is rooted in the AV node, and the repolarization of the atria. The AV nodes includes the bundle of His, which splits into a right and left bundle branch, which stimulates the right and left ventricles of the heart, to contract, respectively. Specifically, each bundle branch spreads to several Purkinje fibers, which cause distinct group of ventricular muscle cells to contract. On the ECG, the contraction of the ventricles of the heart is seen in the QRS complex. Finally, the ventricles must be repolarized which are seen in the J point, ST-segment, T and U-waves on the ECG.

The potential difference between a test electrode, measuring the action potential generated by the heart, and a reference electrode, forms an electrical signal. Upon detection of such an electrical signal generated by the heart, the signal is transmitted to a wireless transmitter incorporated into the electrode patch.

C. Wireless Transmitter

The electrode patch further comprises a wireless transmitter module in addition to an electrode and a power module. The wireless transmitter module works cooperatively with the electrode to receive electronic signals acquired by the electrode from the heart of the subject.

In an aspect, the wireless transmitter module may comprise an application specific integrated circuit, a processor or other circuit, a plurality of signal channels, a multiplexer, an analog to digital converter (ADC), a controller, and a radio. In a further aspect, the wireless transmitter module may include different combinations or fewer of the components described above.

In an aspect, each electrode channel may comprise a filter, an amplifier, a Nyquist filter, and a track and hold circuit. The filter comprises a low pass filter for removing electromagnetic interference signals. The amplifier enhances signals from the electrodes. The Nyquist filter comprises a low pass irrelevant high frequency noise content of the amplified electric signals. Such a filter functions to enhance the reliability of the data generated and avoid measurement error. The track and hold circuit allows the system to sample from each of the channels used at the same time and avoids the potential for error when the signals from each of the channels are combined and displayed for data interpretation.

In an aspect, the multiplexer selects signals sequentially from the electrode channels using time division multiplexing. A person of ordinary skill in the art will recognize that other combination functions can be used.

In an aspect, the ADC is used to convert combined analog signals to digital signals from transmission to the receiver. In an aspect, data from the ADC may be transmitted to a device via a wireless connection. In an aspect, WiFi may be used as a wireless connection. In an alternative aspect, Bluetooth™ may be used as a wireless connection. This disclosure is not intended to limit the various wireless methods to be used in transmitting data from the ADC to a device.

In an aspect, the controller may comprise a digital signal processor (DSP) that decimates the digitized signals to reduce the bandwidth necessary to transmit the electrical signal generated from the heart of the subject.

In an aspect, the radio modulates the converted digital signals with a carrier signal for transmission to the receiver.

D. Power Module

As described above, the electrode patch further comprises a power module in addition to an electrode and wireless transmitter module. The power module provides power to the wireless electrode patch to enable detection and transmission of electrical signals from the subject to the receiver of the cardiac monitoring system.

In an aspect, the power module is configured to supply electrical energy to the electrode sensor. In a further aspect, the power module is configured to supply electrical energy to the wireless transmitter module. In still a further aspect, the power module is configured to supply electrical energy to each of the electrode sensor and the wireless transmitter module. In effect, the power module is configured to supply electrical energy to the whole of the electrode patch.

In an aspect, the wireless cardiac monitoring system comprises a power switch to activate and deactivate the power module of any number of desired electrode patches to be used on a subject during a given time period. Thus, a power switch may activate or deactivate one, two, three, four, five, six, seven, eight, nine, ten, eleven, or even twelve electrode patches.

In an aspect, the power module is designed to house a plurality of batteries. In an alternative aspect, the module utilizes a duty cycle to provide electricity and power to the system.

II. Wireless Receiver

As described above, the wireless cardiac monitoring system further comprises a wireless receiver in addition to a plurality of wireless electrode patches.

In an aspect the wireless receiver comprises a radio, a controller, a digital to analog converter (DAC), a demultiplexer, a transceiver, and a plurality of electrode signal channels.

The radio functions to demodulate received signals for identifying data generated from the combined electrode signals originating from the various electrode patches located at different locations on the subject.

The controller functions to control operation of the various components of the receiver including the ability to control or further process signals from the radio. In an aspect, the controller may convert received signals to digital information or interpolate data transmitted from the electrode patches. Such functions are exemplary, but are in no way meant to be an exhaustive list of operations a controller may perform.

In an aspect, the controller interpolates signals from the electrode patches to return the effective sample rate from about 25 hertz (Hz) to about 1 kilohertz (kHz) or another frequency.

The DAC functions to convert digital signals to analog signals.

The demultiplexer functions to separate the individually regenerated signals onto a separate electrode signal channel for each regenerated signal. Thus, a regenerated signal will be separated onto an electrode signal channel for each of the electrode patches generating data from the heart of the subject.

The transceiver functions to both transmit and receive signals in accordance with communicated with the wireless transmitter module.

In an aspect, the wireless receiver has as many electrode signal channels as there are wireless electrode patches. That is, for every electrode patch used on a subject, the wireless receiver has a corresponding electrode signal channel.

The electrode signal channel comprises a sample and hold circuit, a filter, and an attenuator.

The sample and hold circuit is operated by the controller such that the converted electrode signals from each of the wireless electrode patches appear concurrently on each of the electrode signal channels.

The filter may comprise a low pass reconstruction filter operating to remove high frequency noise associated with the DAC or other conversion process.

The attenuator comprises an amplifier used to reduce the amplitude of the electrode signals to a level associated with electrode signals previously amplified by the transmitter module.

In an aspect, the receiver may be attached to the subject undergoing the cardiac monitoring. Attachment to the subject may comprise wiring, cables, etc.

In a further aspect, the receiver may be close to the body of the subject, but not attached.

Display Module

Upon receipt of the electrical signals from the system, the signals are converted to readable data and presented on a medium. In an aspect, the readable data to be presented on a medium is a rendering of a heart and the cardiac activity of a subject. Such a rendering displays the entire image of the heart so as to give a full view of the cardiac activity of the subject.

In a further aspect, the data presented on a medium is to be interpreted by health care professionals or the subject undergoing measurement. In alternative aspects, such data may be analyzed and interpreted by various healthcare or medical workers with an interest in the cardiac activity of the measured subject.

In several aspects, signals are transmitted to wireless devices and converted into data to be analyzed and interpreted. In an aspect, such data may be transmitted wirelessly for analysis and interpretation to a smart phone. In a further aspect, data from the system may be transmitted and presented on a PC. In yet a further aspect, such data from the system by may be transmitted and presented on a tablet, or any other type of personal electronic device used for data storage and/or presentation.

Placement of the Wireless Electrode Patches

As described above, the present disclosure relates to a wireless cardiac monitoring system including multiple wireless electrode patches. In an aspect, the system comprises three electrode patches. In a further aspect, the system may comprise four electrode patches. In still a further aspect, the system comprises five electrode patches. In yet a further aspect, the system may comprise six electrode patches. In still further aspects, the system may comprise seven, eight, nine, or ten patches.

Traditional cardiac monitoring via electrocardiography utilizes at least 10 electrodes placed at different locations to obtain the most accurate information about the structure and function of the heart of the subject. However, using the systems of the present disclosure, wireless electrode patches may be selectively placed on a patient to determine a complete ECG using a customized (e.g., minimized) number of electrodes. A complete ECG may be defined as an ECG readout or trace representing a normal sinus rhythm and may comprise at least a discernable P wave, QRS complex, and T wave. Additionally, the complete ECG may comprise PR interval, J-point, ST segment, and U wave. It is understood that other portions of the ECG may be includes such as a corrected QT interval. It is further understood that noise or artifacts may be represented in the ECG trace and may be distinguished from the complete trace, as defined above. Additionally or alternatively, a complete ECG may be represented by one or more predetermined characteristic traces such as arrhythmias, including for example, characteristic traces representing atrial fibrillation, atrial flutter, ventricular flutter, and/or ventricular tachycardia. Other characteristic traces may be known and may be catalogued for comparison to determine a discernable complete ECG representative of a match to a characteristic trace.

In order to determine the select number of electrodes and placement of the select number of electrodes in a customized manner, various learning mechanism may be used. For example, heuristics, machine learning, historical patient data, and other learning mechanisms may be used to determine a select number and placement of the wireless electrode patches of the present disclosure. The select number of wireless electrode patches may be optimized to be the minimum number of wireless electrode patches required to produce a complete ECG trace. In certain aspects, the select number of wireless electrode patches may be less than the conventional 12 leads or 10 placed electrodes. As such, the form factor of the wireless electrode patches and the minimized number of the wireless electrode patches provide a complete ECG will minimize intrusiveness to the patient.

Electrodes may be placed at a location on the right arm of the subject (RA), the same location on the left arm of the subject (LA), the right calf (RL), the same location of the left calf (LL), in the fourth intercostal space between rib 4 and rib 5 and immediately to the right of the sternum of the subject (V₁), in the fourth intercostal space between rib 4 and rib 5 and immediately to the left of the sternum of the subject (V₂), between V₁ and V₂ (V₃), in the fifth intercostal space between ribs 5 and 6 in the mid-clavicular line (V₄), horizontally even with V₄ in the left anterior axillary line (V₅), and horizontally even with V₄ and V₅ in the midaxillary line (V₆).

The wireless cardiac monitoring system of the present disclosure utilizes greater than three to five wireless electrode patches to monitor the structural and functional characteristics of the heart of a subject. In an aspect, each of these wireless electrode patches may be placed at a location correlating to any one of RA, LA, RL, LL, and V₁-V₆. In an aspect, the electrode patches may be placed at the RA, the LA, and the LL.

In an aspect, six wireless electrode patches may be placed at various locations on the subject. In a further aspect, the six wireless electrode patches may be placed at (i) a location in the fourth intercostal space between rib 4 and rib 5 and immediately to the right of the sternum of the subject (V₁), (ii) a location in the fourth intercostal space between rib 4 and rib 5 and immediately to the left of the sternum of the subject (V₂), (iii) a location between V₁ and V₂ (V₃), (iv) a location in the fifth intercostal space between ribs 5 and 6 in the mid-clavicular line (V₄), (v) a location horizontally even with V₄ in the left anterior axillary line (V₅), and (vi) a location horizontally even with V₄ and V₅ in the midaxillary line (V₆).

In an aspect, ten wireless electrode patches may be placed at various locations on the subject. In a further aspect, the ten wireless electrode patches may be placed at (i) a location on the right arm of the subject (RA), (ii) the same location on the left arm of the subject (LA), (iii) a location on the right calf (RL), (iv) the same location of the left calf (LL), (v) a location in the fourth intercostal space between rib 4 and rib 5 and immediately to the right of the sternum of the subject (V₁), (vi) a location in the fourth intercostal space between rib 4 and rib 5 and immediately to the left of the sternum of the subject (V₂), (vii) a location between V₁ and V₂ (V₃), (viii) a location in the fifth intercostal space between ribs 5 and 6 in the mid-clavicular line (V₄), (ix) a location horizontally even with V₄ in the left anterior axillary line (V₅), and (x) a location horizontally even with V₄ and V₅ in the midaxillary line (V₆).

In an aspect, between three and five wireless electrode patches may be placed at various locations on the subject. In a further aspect, the plurality of wireless electrode patches may be placed at any of three to five locations including, but not limited to (i) a location in the fourth intercostal space between rib 4 and rib 5 and immediately to the right of the sternum of the subject (V₁), (ii) a location in the fourth intercostal space between rib 4 and rib 5 and immediately to the left of the sternum of the subject (V₂), (iii) a location between V₁ and V₂ (V₃), (iv) a location in the fifth intercostal space between ribs 5 and 6 in the mid-clavicular line (V₄), and (v) a location horizontally even with V₄ in the left anterior axillary line (V₅).

In an aspect, the wireless cardiac monitoring system may be used to measure other vital medical characteristics including body temperature. In a further aspect, the wireless cardiac monitoring system may be used to measure pulse rate. In still a further aspect, the wireless cardiac monitoring system may be used to measure heart rate. In yet a further aspect, the wireless cardiac monitoring system may be used to measure respiration rate. In another aspect, the wireless cardiac monitoring system may be used to measure EEG signals. In still another aspect, the wireless cardiac monitoring system may be used to measure pulse oximeter signals.

In addition to improving comfort and overall appearance of the subject undergoing cardiac monitoring, the overall quality of data is comparable between the present disclosure comprising fewer electrodes and a traditional 12-lead electrocardiogram.

Aspects

Aspect 1. A medical monitoring system comprising a plurality of wireless electrode patches, each of the wireless electrode patches comprising a sensor configured to detect a medical characteristic of a subject, a wireless module configured to transmit a signal indicative of the detected medical characteristic, and a power module configured to supply electrical energy to one or more of the sensor and the wireless module; and a wireless receiver configured to communicate with each of the wireless electrode patches to receive at least the signal indicative of the detected medical characteristic.

Aspect 2. The medical monitoring system of aspect 1, wherein the detected medical characteristic is a rendering of a heart of the subject.

Aspect 3. The medical monitoring system of aspect 2, wherein the rendering of the heart of the subject displays a complete and entire image of the heart.

Aspect 4. The medical monitoring system of aspect 3, wherein the plurality of wireless electrode patches comprises fewer than 12 wireless electrode patches.

Aspect 5. The medical monitoring system of aspect 4, wherein the plurality of wireless electrode patches comprises six wireless electrode patches.

Aspect 6. The medical monitoring system of aspect 5, wherein the location of the six wireless electrode patches includes a location in the fourth intercostal space between rib 4 and rib 5 and immediately to the right of the sternum of the subject (V1), a location in the fourth intercostal space between rib 4 and rib 5 and immediately to the left of the sternum of the subject (V2), a location between V1 and V2 (V3), a location in the fifth intercostal space between ribs 5 and 6 in the mid-clavicular line (V4), and a location horizontally even with V4 in the left anterior axillary line (V5), and a location horizontally even with V4 and V5 in the midaxillary line (V6).

Aspect 7. The medical monitoring system of Aspect 4, wherein the plurality of wireless electrode patches comprises ten wireless electrode patches.

Aspect 8. The medical monitoring system of aspect 7, wherein the location of the ten wireless electrode patches includes a location on the right arm of the subject (RA), the same location on the left arm of the subject (LA), a location on the right calf (RL), the same location of the left calf (LL), a location in the fourth intercostal space between rib 4 and rib 5 and immediately to the right of the sternum of the subject (V1), a location in the fourth intercostal space between rib 4 and rib 5 and immediately to the left of the sternum of the subject (V2), a location between V1 and V2 (V3), a location in the fifth intercostal space between ribs 5 and 6 in the mid-clavicular line (V4), a location horizontally even with V4 in the left anterior axillary line (V5), and a location horizontally even with V4 and V5 in the midaxillary line (V6).

Aspect 9. The medical monitoring system of aspect 4, wherein the plurality of wireless electrode patches comprises between three and five wireless electrode patches.

Aspect 10. The medical monitoring system of aspect 9, wherein the location of the between three and five wireless electrode patches includes a location in the fourth intercostal space between rib 4 and rib 5 and immediately to the right of the sternum of the subject (V1), a location in the fourth intercostal space between rib 4 and rib 5 and immediately to the left of the sternum of the subject (V2), a location between V1 and V2 (V3), a location in the fifth intercostal space between ribs 5 and 6 in the mid-clavicular line (V4), and a location horizontally even with V4 in the left anterior axillary line (V5).

Aspect 11. The medical monitoring system of any one of aspects 1 to 10, wherein the detected medical characteristic is pulse rate.

Aspect 12. The medical monitoring system of any one of aspects 1 to 10, wherein the detected medical characteristic is heart rate.

Aspect 13. The medical monitoring system of any one of aspects 1 to 10, wherein the detected medical characteristic is respiration rate.

Aspect 14. The medical monitoring system of any one of aspects 1 to 10, wherein the detected medical characteristic is body temperature.

Aspect 15. The medical monitoring system of any one of aspects 1 to 10, wherein the detected medical characteristic is EEG signaling.

Aspect 16. The medical monitoring system of any one of aspects 1 to 10, wherein the detected medical characteristic is pulse oximeter signaling.

Aspect 17. The medical monitoring system of any one of aspects 1 to 16, wherein the wireless electrode patch comprises a thermoplastic substrate material.

Aspect 18. The medical monitoring system of aspect 17, wherein the thermoplastic substrate material is a flexible silicone material.

Aspect 19. The medical monitoring system of any one of aspects 1 to 16, wherein the wireless electrode patch comprises a silver-silver chloride material.

Aspect 20. The medical monitoring system of any one of aspects 1 to 19, wherein the wireless electrode patches may be labeled by location, color-coded, or both.

Aspect 21. The medical monitoring system of any one of aspects 1 to 20, wherein the system generates and outputs data corresponding to the detected medical characteristic to a display.

Aspect 22. The medical monitoring system of aspects 21, wherein the display is one of a tablet, a smart phone, a PC, or a personal electronic device.

Aspect 23. The medical monitoring system of any one of aspects 1 to 20, wherein the system generates and outputs data corresponding to the detected medical characteristic to a memory device.

Aspect 24. A medical monitoring system comprising: a plurality of wireless electrode patches, each of the wireless electrode patches comprising a sensor configured to detect a medical characteristic of a subject, a wireless module configured to transmit a signal indicative of the detected medical characteristic, and a power module configured to supply electrical energy to one or more of the sensor and the wireless module, wherein the plurality of wireless electrode patches comprises fewer than 10 wireless electrode patches, and wherein each of the plurality of wireless electrode patches has a separate form factor that is spaced from the form factors of the other electrode patches; and a wireless receiver configured to communicate with each of the wireless electrode patches to receive at least the signal indicative of the detected medical characteristic and to generate an output based on the received signal.

Aspect 25. The medical monitoring system of aspect 24, wherein the detected medical characteristic comprises one or more electrical signals indicative of an activity of a heart of a user of the medical monitoring system.

Aspect 26. The medical monitoring system of any one of aspects 24-25, wherein the output comprises a complete electrocardiogram trace.

Aspect 27. The medical monitoring system of any one of aspects 24-26, wherein a placement of the plurality of wireless electrode patches is customized for a user of the medical monitoring system.

Aspect 28. The medical monitoring system of any one of aspects 24-27, wherein the plurality of wireless electrode patches comprises six wireless electrode patches.

Aspect 29. The medical monitoring system of aspect 28, wherein the location of the six wireless electrode patches includes a location in the fourth intercostal space between rib 4 and rib 5 and immediately to the right of the sternum of the subject (V1), a location in the fourth intercostal space between rib 4 and rib 5 and immediately to the left of the sternum of the subject (V2), a location between V1 and V2 (V3), a location in the fifth intercostal space between ribs 5 and 6 in the mid-clavicular line (V4), and a location horizontally even with V4 in the left anterior axillary line (V5), and a location horizontally even with V4 and V5 in the midaxillary line (V6).

Aspect 30. The medical monitoring system of any one of aspects 24-29, wherein each of the plurality of wireless electrode patches comprises a thermoplastic substrate material.

Aspect 31. The medical monitoring system of aspect 30, wherein the thermoplastic substrate material is a flexible silicone material.

Aspect 32. The medical monitoring system of any one of aspects 24-31, wherein each of the plurality of wireless electrode patches comprises a silver-silver chloride material.

Aspect 33. A method of determining a medical characteristic of a user, the method comprising: determining a number of wireless electrode patches to be placed on the user, each of the wireless electrode patches comprising a sensor configured to detect a medical characteristic of a subject, a wireless module configured to transmit a signal indicative of the detected medical characteristic, and a power module configured to supply electrical energy to one or more of the sensor and the wireless module, wherein the number is less than 10; determining a placement position on the user of each of the wireless electrode patches, wherein the number of the wireless electrode patches and the placement position of each of the wireless electrode patches is configured to facilitate generation of a complete electrocardiogram trace using a minimal number of the wireless electrode patches; disposing the number of the wireless electrode patches at the determined placement positions on the user; receiving, wirelessly, one or more signals indicative of an activity of the heart of the user; causing generation of the complete electrocardiogram trace based on the received one or more signals.

Aspect 34. The method of aspect 33, wherein each of the plurality of wireless electrode patches has a separate form factor, and wherein the placement positions are configured such that the form factor of each of the wireless electrode patches is spaced from the form factors of the other electrode patches.

Aspect 35. The method of any one of aspects 33-34, wherein determining a number of the wireless electrode patches is based on a learning mechanism.

Aspect 36. The method of aspect 35, wherein the learning mechanism comprises heuristics, machine learning, or historical user medial data, or a combination thereof.

Aspect 37. The method of any one of aspects 33-36, wherein determining a placement position on the user of the wireless electrode patches is based on a learning mechanism.

Aspect 38. The method of aspect 37, wherein the learning mechanism comprises heuristics, machine learning, or historical user medial data, or a combination thereof.

Aspect 39. The method of any one of aspects 33-38, wherein the number of wireless electrode patches is six.

Aspect 40. The method of aspect 39, wherein the placement positions of the six wireless electrode patches includes a location in the fourth intercostal space between rib 4 and rib 5 and immediately to the right of the sternum of the subject (V1), a location in the fourth intercostal space between rib 4 and rib 5 and immediately to the left of the sternum of the subject (V2), a location between V1 and V2 (V3), a location in the fifth intercostal space between ribs 5 and 6 in the mid-clavicular line (V4), and a location horizontally even with V4 in the left anterior axillary line (V5), and a location horizontally even with V4 and V5 in the midaxillary line (V6).

Aspect 41. The method of any one of aspects 33-40, wherein each of the plurality of wireless electrode patches comprises a thermoplastic substrate material.

Aspect 42. The method of aspect 41, wherein the thermoplastic substrate material is a flexible silicone material.

Aspect 43. The method of any one of aspects 33-42, wherein each of the plurality of wireless electrode patches comprises a silver-silver chloride material.

### EXAMPLES

In an aspect, the wireless cardiac monitoring system is used to measure structural and functional medical characteristics of the heart of a subject. First, a plurality of wireless electrode patches is placed at various anatomical locations on the skin of a subject. Such locations may include, but are not limited to no fewer than three of a location on the right arm of the subject (RA), the same location on the left arm of the subject (LA), the right calf (RL), the same location of the left calf (LL), in the fourth intercostal space between rib 4 and rib 5 and immediately to the right of the sternum of the subject (V₁), in the fourth intercostal space between rib 4 and rib 5 and immediately to the left of the sternum of the subject (V₂), between V₁ and V₂ (V₃), in the fifth intercostal space between ribs 5 and 6 in the mid-clavicular line (V₄), horizontally even with V₄ in the left anterior axillary line (V₅), and horizontally even with V₄ and V₅ in the midaxillary line (V₆).

Upon securing the plurality of wireless electrode patches to the skin of the subject, measurement of medical characteristics may begin. Each individual wireless electrode patch contains an electrode sensor designed to detect electrical signals from each contraction, or beat of the heart of a subject. The individual wireless electrode patch comprises a wireless transmitter module and a power module in addition to the electrode sensor. Cardiac monitoring and measurement of characteristics may begin upon activation of the wireless electrode patch through the power module supplying electrical energy to the entirety of the patch. Electrical signals generated by the heart of the subject are detected by the electrode sensor of the monitoring system and transfers these signals to the wireless transmitter module portion of the wireless electrode patch. Upon detection of electrical signals from the heart, the transmitter module processes the signaling in a variety of ways before relaying the electrical signal to the wireless receiver via radio transmission. Such radio transmission occurs between the wireless transmitter module and the wireless receiver of the cardiac monitoring system. Upon receiving the signaling from the transmitter, the wireless receiver processes, filters, and converts the electrical signals from the heart of the patient from raw data into a comprehensible format for review by a healthcare professional or by the subject himself.

In some aspects the systems, patches, sensors, and associated components described herein are suitable for use in any applicable medical and/or healthcare-related application. Exemplary applications include, but are not limited to, general healthcare delivery, diagnostic applications, therapeutic applications, and drug delivery applications.

It is to be understood that any feature descripted in relation to any one aspect or Example may be used alone, or in combination with other features described, and may also be used in combination with one or more features of any other of the aspects or Examples, or any combination of any other of the aspects and examples. Further, equivalents and modifications not described above may also be employed without departing from the scope of the disclosure, which is defined in the accompanying claims.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein can be different from the actual publication dates, which can require independent confirmation.

1. A medical monitoring system comprising;
a plurality of wireless electrode patches, each of the wireless electrode patches comprising a sensor configured to detect a medical characteristic of a subject, a wireless module configured to transmit a signal indicative of the detected medical characteristic, and a power module configured to supply electrical energy to one or more of the sensor and the wireless module, wherein the plurality of wireless electrode patches comprises fewer than 10 wireless electrode patches, and wherein each of the plurality of wireless electrode patches has a separate form factor that is spaced from the form factors of the other electrode patches; and
a wireless receiver configured to communicate with each of the wireless electrode patches to receive at least the signal indicative of the detected medical characteristic and to generate an output based on the received signal.

2. The medical monitoring system of claim 1, wherein the detected medical characteristic comprises one or more electrical signals indicative of an activity of a heart of a user of the medical monitoring system.

3. The medical monitoring system of claim 1, wherein the output comprises a complete electrocardiogram trace.

4. The medical monitoring system of claim 1, wherein a placement of the plurality of wireless electrode patches is customized for a user of the medical monitoring system.

5. The medical monitoring system of claim 1, wherein the plurality of wireless electrode patches comprises six wireless electrode patches.

6. The medical monitoring system of claim 5, wherein each of the six wireless electrode patches includes: a location in a fourth intercostal space between rib 4 and rib 5 and immediately to the right of a sternum of the subject (V₁); a location in the fourth intercostal space between rib 4 and rib 5 and immediately to the left of the sternum of the subject (V₂); a location between V₁ and V₂ (V₃), a location in a fifth intercostal space between ribs 5 and 6 in a mid-clavicular line (V₄); a location horizontally even with V₄ in a left anterior axillary line (V₅); and a location horizontally even with V₄ and V₅ in a midaxillary line (V₆).

7. The medical monitoring system of claim 1, wherein each of the plurality of wireless electrode patches comprises a thermoplastic substrate material.

8. The medical monitoring system of claim 7, wherein the thermoplastic substrate material is a flexible silicone material.

9. The medical monitoring system of claim 1, wherein each of the plurality of wireless electrode patches comprises a silver-silver chloride material.

10. A method of determining a medical characteristic of a user, the method comprising;
determining a number of wireless electrode patches to be placed on the user, each of the wireless electrode patches comprising a sensor configured to detect a medical characteristic of a subject, a wireless module configured to transmit a signal indicative of the detected medical characteristic, and a power module configured to supply electrical energy to one or more of the sensor and the wireless module, wherein the number is less than 10;
determining a placement position on the user of each of the wireless electrode patches, wherein the number of the wireless electrode patches and the placement position of each of the wireless electrode patches is configured to facilitate generation of a complete electrocardiogram trace using a minimal number of the wireless electrode patches;
disposing the number of the wireless electrode patches at the determined placement positions on the user;
receiving, wirelessly, one or more signals indicative of an activity of the heart of the user;
causing generation of the complete electrocardiogram trace based on the received one or more signals.

11. The method of claim 10, wherein each of the wireless electrode patches has a separate form factor, and wherein the placement positions are configured such that the form factor of each of the wireless electrode patches is spaced from the form factors of the other electrode patches.

12. The method of claim 10, wherein determining a number of the wireless electrode patches is based on a learning mechanism.

13. The method of claim 12, wherein the learning mechanism comprises heuristics, machine learning, or historical user medial data, or a combination thereof.

14. The method of claim 10, wherein determining a placement position on the user of the wireless electrode patches is based on a learning mechanism.

15. The method of claim 14, wherein the learning mechanism comprises heuristics, machine learning, or historical user medial data, or a combination thereof.

16. The method of claim 10, wherein the number of wireless electrode patches is six.

17. The method of claim 16, wherein placement positions of the six wireless electrode patches include: a location in a fourth intercostal space between rib 4 and rib 5 and immediately to the right of a sternum of a subject (V₁); a location in the fourth intercostal space between rib 4 and rib 5 and immediately to the left of the sternum of the subject (V₂); a location between V₁ and V₂ (V₃); a location in a fifth intercostal space between ribs 5 and 6 in a mid-clavicular line (V₄); a location horizontally even with V₄ in a left anterior axillary line (V₅); and a location horizontally even with V₄ and V₅ in a midaxillary line (V₆).

18. The method of claim 10, wherein each of the wireless electrode patches comprises a thermoplastic substrate material.

19. The method of claim 18, wherein the thermoplastic substrate material is a flexible silicone material.

20. The method of claim 10, wherein each of the wireless electrode patches comprises a silver-silver chloride material.
